(19) United States
(12) Patent Application Publication
(10) Pub. No.: US 2026/0233001 A1
(43) Pub. Date: Aug. 13, 2026

(54) TASTE PRESENTATION DEVICE AND TASTE PRESENTATION METHOD

(71) Applicants: KIRIN HOLDINGS KABUSHIKI KAISHA, Tokyo (JP); MEIJI UNIVERSITY, Tokyo (JP)

(72) Inventors: Ai TSUJI SATO, Nakano-ku (JP); Homei MIYASHITA, Nakano-ku (JP)

(73) Assignees: KIRIN HOLDINGS KABUSHIKI KAISHA, Tokyo (JP); MEIJI UNIVERSITY, Tokyo (JP)

(21) Appl. No.: 19/155,761

(22) PCT Filed: Mar. 7, 2024
(86) PCT No.: PCT/JP2024/008859

§ 371 Date: Aug. 12, 2025

(30) Foreign Application Priority Data
Feb. 17, 2023 (JP) 2023-023226

(51) Int. Cl. A61N 1/36; A61M 21/00; A61N 1/04

(52) U.S. Cl. CPC A61N 1/36025; A61M 21/00; A61N 1/0456; A61N 1/36034; A61M 2021/0072

(57) ABSTRACT

In a taste presentation device may include: a first electrode and a second electrode capable of forming an electric circuit between a food or drink to be ingested by a user and a body of the user; and electrical stimulation generator supplying a current to generate electrical stimulation between the first electrode and the second electrode. The electrical stimulation generator may start the supply of the current in a state of cathode stimulation in which the second electrode is a cathode, and then to change a direction of the current so as to switch to a state of anode stimulation in which the second electrode is an anode. In the cathode stimulation, a holding time Tx1 during which the current is to be held at a setting value Is1 of the current in the cathode stimulation may be set to 0.30 seconds or more.

### TECHNICAL FIELD

The present invention relates to a taste presentation device or the like for giving a change in taste sensed by a user using electrical stimulation.

### BACKGROUND ART

As techniques for enhancing a salty taste or the like and enhancing a sense of satisfaction for users who have no choice but to take a lightly seasoned meal with limited salt or sugar due to circumstances in health management or the like, techniques in which a taste of a food or drink is modified and presented to match a user's preference and the like using electrical stimulation have been proposed. For example, there is proposed a taste presentation device that supplies a rectangular wave current with a constant frequency between an anode in contact with a body part, such as the back of the neck, of a user and a cathode in contact with a food or drink to produce an enhancing effect with respect to the user's taste (see Patent Literature 1). There is also proposed a device that supplies a two-phase current to an electrode disposed to be in contact with a tongue or the like of a user to electrically stimulate a nerve branch that carries taste sensations to the brain (see Patent Literature 2).

### CITATION LIST

### Patent Literature

Patent Literature 1: JP2018-42991A
Patent Literature 2: JP2010-517675A

### SUMMARY OF INVENTION

### Technical Problem

When modification such as enhancement of a salty taste is attempted using electrical stimulation, a user may feel a sense of discomfort or sense an electric taste. The sense of discomfort means, for example, an unnatural sensation felt by the user accompanying the electrical stimulation, such as an unnatural sensation felt by the user due to a time difference from when a food or drink is put in the mouth to when a modified taste is sensed, or an unnatural sensation that may be felt in a body part close to an electrode when a current flows. The electric taste means a metallic taste or a sour taste felt when the current flows. If a current value is decreased in order to avoid such inconvenience, a salty taste enhancing effect is accordingly weakened, and there is a possibility that it becomes difficult for the user to sense the effect.

Therefore, an object of the present invention is to provide a taste presentation device and a taste presentation method capable of exhibiting a taste modification effect to be sensed by a user while suppressing a current value to be relatively small.

### Solution to Problem

A taste presentation device according to an aspect of the present invention includes: a first electrode and a second electrode provided to be capable of forming an electric circuit between a food or drink to be ingested by a user and a body of the user; and electrical stimulation generation means supplying a current to generate electrical stimulation between the first electrode and the second electrode, wherein the electrical stimulation generation means is provided to start the supply of the current in a state of cathode stimulation in which the second electrode is a cathode, and then to change a direction of the current so as to switch to a state of anode stimulation in which the second electrode is an anode, and in the cathode stimulation, a holding time during which the current is to be held at a setting value of the current in the cathode stimulation is set to 0.30 seconds or more.

A taste presentation method according to an aspect of the present invention includes: a process of providing a first electrode and a second electrode to be capable of forming an electric circuit between a food or drink to be ingested by a user and a body of the user; and a process of supplying a current to generate electrical stimulation between the first electrode and the second electrode, wherein, in the process of supplying the current, the supply of the current is started in a state of cathode stimulation in which the second electrode is a cathode, and then a direction of the current is changed so as to switch to a state of anode stimulation in which the second electrode is an anode, and in the cathode stimulation, a holding time during which the current is to be held at a setting value of the current in the cathode stimulation is set to 0.30 seconds or more.

### BRIEF DESCRIPTION OF DRAWINGS

FIG. 1 is a diagram illustrating an example of a taste presentation device according to an embodiment of the present invention.

FIG. 2 is a diagram illustrating another example of the taste presentation device.

FIG. 3 is a diagram illustrating a configuration example of an electrical stimulation generation unit.

FIG. 4 is a diagram illustrating an example of a current waveform supplied from an electrical stimulation generation unit.

### DESCRIPTION OF EMBODIMENTS

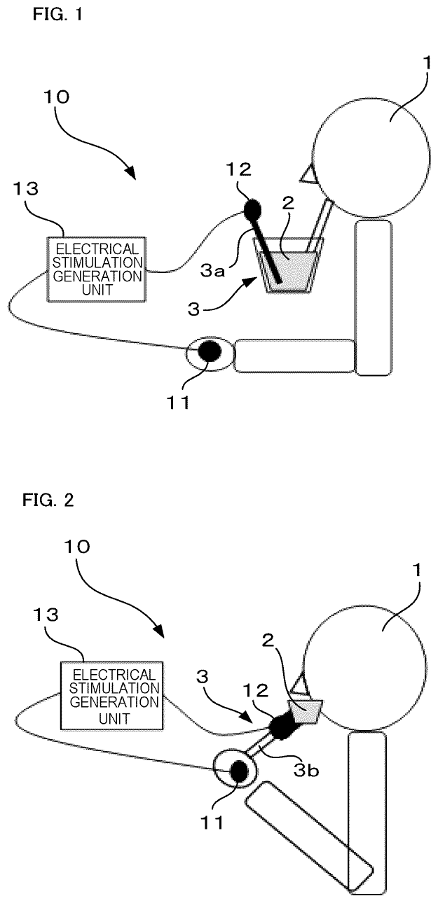

FIG. 1 illustrates an example of a taste presentation device according to an embodiment of the present invention. A taste presentation device 10 in FIG. 1 includes a first electrode 11 provided to be in contact with the user 1, a second electrode 12 provided to be in contact with a food or drink 2 ingested by the user 1, and an electrical stimulation generation unit 13 as an example of electrical stimulation means for supplying a current to generate electrical stimulation between the electrodes 11 and 12.

As an example, the first electrode 11 is attached to a hand or an arm of the user 1. A gel electrode may be used as the first electrode 11 and attached to the user, or the first electrode 11 may be provided to be held by the user's hand. As an example, the second electrode 12 is attached to conductive tableware 3 used by the user for eating and drinking. The tableware 3 itself may be used as the second electrode 12. For example, as illustrated in FIG. 1, the second electrode 12 may be attached to a muddler 3a immersed in a beverage as an example of the food or drink 2, or the muddler 3a itself may be used as the second electrode 12. Alternatively, as illustrated in FIG. 2, the second electrode 12 may be attached to an instrument (including so-called cutlery) 3b such as a fork, a spoon, or a pair of chopsticks used for ingesting food as an example of the food or drink 2, or those instruments 3b may be used as the second electrodes 12. In any case, the first electrode 11 and the second electrode 12 may be configured in any appropriate configuration as long as an electric circuit is formed between the food or drink 2 to be ingested by the user 1 and a body of the user 1 such that a current for applying electrical stimulation flows through an organ such as a tongue with which the user 1 senses a taste. The tableware 3 to which the second electrode 12 is to be attached is not limited to the examples of FIGS. 1 and 2, and may be any object to which the second electrode 12 is to be attached, such as a dish, a wooden bowl, a tea bowl, a rice bowl, a cup, a glass, a mug, a goblet, a sake cup, a bowl, a glass mug, a tea cup, a tumbler, a water bottle, a pair of chopsticks, a fork, a spoon, a knife, a wooden spoon, a skewer, a toothpick, or a straw.

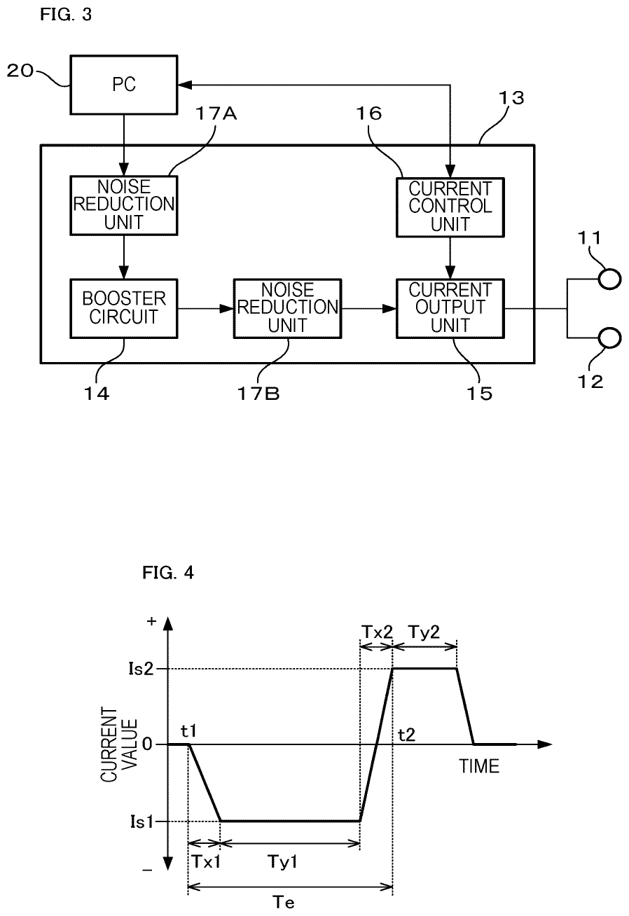

As illustrated in FIG. 3, an electrical stimulation generation unit 13 includes a booster circuit 14, a current output unit 15, a current control unit 16, and noise reduction units 17A and 17B (represented by reference numeral 17 in some cases). For example, the booster circuit 14 boosts a direct current supplied from an external power source (not illustrated) via a personal computer (PC) 20 to a predetermined voltage and supplies the direct current to the current output unit 15. The external power source is, for example, an AC commercial power source. The current from the power source is not limited to the example supplied via the PC 20, and may be supplied via an appropriate converting device having an AC-DC conversion function, such as a converter, an adapter, or the like. A DC power source such as a power battery may be used as the external power source.

The current output unit 15 shapes a current waveform according to a command value provided from the current control unit 16 and supplies the current waveform to the electrodes 11 and 12. The current control unit 16 is configured as, for example, a computer unit using a microprocessor, generates the command value of the current waveform according to setting information of the current waveform provided from the PC 20, and outputs the command value to the current output unit 15 to control the current supplied to the electrodes 11 and 12. For example, the current control unit 16 can control a direction of the current such that anode stimulation in which the second electrode 12 is an anode and cathode stimulation in which the second electrode 12 is a cathode can be selected, and can control a magnitude (current value) and a rate of change of the current to target values given from the PC 20. An example of the current control will be described later.

The noise reduction units 17 reduce noise components included in the current supplied between the electrodes 11 and 12. The noise reduction units 17 are examples of noise reduction means. According to study of the inventor, it has been found that a noise component included in a current is a factor that worsening a sense of discomfort and an electric taste, and the sense of discomfort and the electric taste tend to increase as the noise component increases. The noise reduction units 17 are provided to enhance a taste modification effect by suppressing the influence of a minute noise component on the sense of discomfort and the electric taste. Incidentally, the term “reduction” means to relatively reduce the noise component in the current as compared with a case where the noise reduction unit 17 is not provided, and is a concept including not only a case where some noise component remains but also processing such as “removal”, “elimination”, and “mitigation” in which the noise component is reduced to an undetectable level. “Modification” is a concept that means to change a taste sensed by the user 1 from an original taste sensed when no electrical stimulation is added, and “presentation” is a concept that means to make the user 1 sense the modified taste.

The noise reduction units 17 may be configured by various elements, such as a capacitor, an inductor, and a ferrite core, used for reducing noise components in various electric circuits alone or in appropriate combination. In an example of FIG. 3, the noise reduction unit 17A is provided on an input side of the booster circuit 14 for the purpose of reducing a noise component derived from the external power source, and the noise reduction unit 17B is provided on an output side of the booster circuit 14 for the purpose of removing a noise component corresponding to an internal oscillation frequency generated at the time of boosting by the booster circuit 14. However, positions and number of the noise reduction units 17 may be appropriately changed in accordance with an internal configuration such as a position of a noise generation source in the electrical stimulation generation unit 13.

Next, the example of the current control by the current control unit 16 will be described. FIG. 4 illustrates an example of the current waveform controlled by the current control unit 16, where the horizontal axis represents time and the vertical axis represents a current value. The current value is indicated as a positive value when the second electrode 12 is the anode and indicated as a negative value when it is the cathode, respectively. In the following description, a change in a direction in which an absolute value increases is expressed as an “increase” regardless of whether the current value changes in the positive or negative direction.

In the example of FIG. 4, it is assumed that the user 1 ingests the food or drink 2 at time t1 so that the electrodes 11 and 12 are electrically connected, and the electric circuit including the user 1 is formed. When the formation of the electric circuit is detected at the time t1, the current control unit 16 starts to supply a current by setting a direction of the current so as to provide the cathode stimulation, that is, so as to set the first electrode 11 to be the anode and the second electrode 12 to be the cathode, and gradually increases the current value to a setting value Is1 over a predetermined increase time Tx1. The setting value Is1 is a value set as a target value of the current that is to be held in the cathode stimulation. After reaching the setting value Is1, the current control unit 16 holds the setting value Is1 for a predetermined holding time Ty1. When the holding time Ty1 elapses, the current control unit 16 changes the direction of the current such that the first electrode 11 becomes the cathode and the second electrode 12 becomes the anode, and increases the current value of the second electrode 12 to a setting value Is2 over a predetermined reversal time Tx2. The setting value Is2 is a value set as a target value of the current that is to be held in the anode stimulation. After the current value reaches the setting value Is2 at time t2, the current control unit 16 holds the setting value Is2 for a predetermined holding time Ty2, and stops supplying the current when the holding time Ty2 has elapsed.

As in the example of FIG. 4, when the polarity is reversed after the start of the supply of the current, it is possible to sufficiently bring out the action of changing the taste using the polarity reversal while suppressing the setting values Is1 and Is2 of the current to relatively small values, thereby enhancing the taste modification effect. There is a tendency that the cathode stimulation suppresses the sense of discomfort of the user 1 to be relatively weak but has a weak taste modification effect, and the anode stimulation has a relatively strong taste modification effect but induces a relatively strong sense of discomfort or a relatively strong electric taste. Therefore, it is possible to secure a large amount of change in the current value during the reversal and enhance the taste modification effect by performing the cathode stimulation first to suppress the sense of discomfort and then reversing the polarity to perform the anode stimulation. In addition, it is possible to reduce the sense of discomfort and the electric taste by suppressing the setting value Is2 of the current during the anode stimulation to be relatively small.

In the example of FIG. 4, various parameters defining the current waveform, that is, the setting values Is1 and Is2 of the current, the increase time Tx1 until the setting value Is1 is reached, the holding time Ty1 of the setting value Is1 in the cathode stimulation, the reversal time Tx2 until the polarity is reversed from the cathode stimulation to the anode stimulation and the setting value Is2 of the anode stimulation is reached, and the holding time Ty2 of the setting value Is2 in the anode stimulation are set in accordance with an environmental situation such as an individual difference of the user 1, a degree of the intended taste modification effect, or a type of the food or drink 2 to be ingested, and preferable ranges thereof are as follows.

First, the holding time Ty1 of the setting value Is1 of the current in the cathode stimulation will be described. The holding time Ty1 is a parameter that is important in terms of exhibiting the effect of the cathode stimulation since there is a possibility that the effect of performing the cathode stimulation first is less likely to be exhibited if the holding time Ty1 is excessively short. From the viewpoint of exhibiting the effect of the cathode stimulation, the holding time Ty1 may be set to 0.30 seconds or more and preferably 0.50 seconds or more.

On the other hand, if the holding time Ty1 is excessively long, there is a possibility that the exhibition of the taste modification effect using the anode stimulation is delayed with respect to the user's eating and drinking behavior, thereby giving an unnatural sensation. However, there is an individual difference in the user's eating and drinking behavior, and an upper limit of the holding time Ty1 may also change in relation thereto. When a user who is relatively fast in the eating and drinking behavior is targeted, the modification effect needs to be exhibited early in comparison with a user who is slow in the eating and drinking behavior, and thus it is preferable to set the upper limit of the holding time Ty1 relatively short. On the other hand, when the user who is relatively slow in the eating and drinking behavior is targeted, there is a margin in time when the modification effect needs to be exhibited, and thus it is possible to set the upper limit of the holding time Ty1 relatively long.

However, when the increase time Tx1 or the reversal time Tx2 is set to be excessively long, the time required to exhibit the effect by the anode stimulation is prolonged even if only the upper limit of the holding time Ty1 is limited. When the time is prolonged, there may be a problem that a time difference from when food or drink is put in a mouth to when a modified taste is sensed, that is, a time difference between the timing at which the user expects to sense the taste and the timing at which the taste is actually sensed, is widened, which gives the user the sense of discomfort. Hereinafter, such a problem may be referred to as unnaturalness of timing. Therefore, in order to appropriately set the time until manifestation of the anode stimulation, it is preferable to set an upper limit for a total time of the increase time Tx1, the holding time Ty1, and the reversal time Tx2, in other words, an elapsed time Te (=Tx1+Ty1+Tx2) from the time t1 when the supply of the current of the cathode stimulation is started to the time t2 when the current of the anode stimulation reaches the setting value Is2 through the cathode stimulation rather than to limit the upper limit of the holding time Ty1 alone. The holding time Ty1 may be set within such a range of the elapsed time Te. The elapsed time Te may be set to 1.30 seconds or less, preferably 1.20 seconds or less, and more preferably 1.00 seconds or less. A lower limit of the elapsed time Te can be set to 0.70 seconds or more and preferably 0.85 seconds or more as a guide on the premise that the holding time Ty1 is set to the above-described lower limit or more. However, since the effect of the cathode stimulation is greatly affected by the holding time Ty1, it is sufficient to prioritize keeping the elapsed time Te within the above-described upper limit or less while setting the holding time Ty1 to the above-described lower limit or more, and it is not always necessary to strictly limit the lower limit of the elapsed time Te itself.

The increase time Tx1, the holding time Ty1, and the reversal time Tx2 can be appropriately combined also when the lower limit of the holding time Ty1 and the upper limit of the elapsed time Te are set within the above-described ranges. The increase time Tx1 may be set to 0.10 seconds or more, preferably 0.15 seconds or more, and more preferably 0.20 seconds or more as a guide since there is a possibility that the user feels the sense of discomfort with the stimulation if the increase time Tx1 is excessively short. Further, the increase time Tx1 may be set to 0.50 seconds or less from the viewpoint of keeping the elapsed time Te within the above-described range. When the upper limit of the holding time Ty1 is set to approximately 0.80 seconds or less, there is a high possibility that the above-described inconvenience can be avoided even for the user who is fast in the eating and drinking behavior. Furthermore, the reversal time Tx2 may be set to 0.20 seconds or more since there is a possibility that the user feels the sense of discomfort with the stimulation if the reversal time Tx2 is excessively short. Further, the reversal time Tx2 may be set to 0.50 seconds or less from the viewpoint of keeping the elapsed time Te within the above-described range.

Regarding the holding time Ty2 of the setting value Is2 of the current in the anode stimulation, an appropriate range of a time length for which the taste modification effect is to be maintained may be determined in accordance with an environment such as the speed of the user's eating and drinking behavior and a type of food or drink to set the holding time Ty2 in accordance with the determined appropriate range.

Next, the setting values Is1 and Is2 of the current will be described. The setting values Is1 and Is2 of the current may be set such that absolute values thereof are equal to each other or different from each other. The absolute values of the setting values Is1 and Is2 themselves can be appropriately set in accordance with an intended taste modification effect, and, as a guide, may be set in a range of 0.01 mA to 1.00 mA, preferably in a range of 0.05 mA to 0.70 mA, and more preferably in a range of 0.05 mA to 0.50 mA. However, when the absolute values of the setting value Is1 in the cathode stimulation and the setting value Is2 in the anode stimulation themselves are relatively large, there is a possibility that the user is likely to sense the electric taste. Alternatively, when the absolute value of the setting value Is2 in the anode stimulation is equal to or larger than the absolute value of the setting value Is1 in the cathode stimulation, there is a possibility that a relatively large effect of the anode stimulation is exhibited so that the user is likely to sense the electric taste. In order to avoid such inconvenience, the absolute values of the setting values Is1 and Is2 may be set to be equal to each other, and an upper limit of the absolute values may be set to 0.30 mA or less. Further, when the setting values Is1 and Is2 are equally set, the lower limit of the absolute values of the setting values Is1 and Is2 is preferably set to 0.25 mA or more from the viewpoint of exhibiting the taste modification effect. Alternatively, one of preferable workarounds is to set the magnitude relationship between the absolute values of the setting values Is1 and Is2 such that the absolute value of the setting value Is1 in the cathode stimulation is larger than the absolute value of the setting value Is2 in the anode stimulation.

The current waveform illustrated in FIG. 4 is an example, and the waveform may be set in consideration of various items such as a purpose of applying the electrical stimulation, that is, the viewpoint of how to modify the taste and present the modified taste to the user 1, a type of the food or drink 2 that is a target of taste modification, or preferences and other personality of the user 1. In the example of FIG. 4, the current value is changed at a constant increase rate in the increase time Tx1 and the reversal time Tx2, but such current changes may be changes in a quadratic curve shape or other non-linear shapes. Also in the holding times Ty1 and Ty2, the current value may be changed within an allowable range without being fixed to a constant value. For example, the current waveform may be set to an appropriate waveform such as a sinusoidal waveform or a sawtooth waveform. Even if such a change is given, it can be regarded as the setting value of the current in each of the cathode stimulation and the anode stimulation as long as it can be recognized as being set as a target value of the current for applying the cathode stimulation and the anode stimulation.

In the above embodiment, the effect of reducing the sense of discomfort is achieved by providing the noise reduction means, but the noise reduction means is not necessarily essential. The noise reduction means may be omitted when the influence of a noise component is relatively small. Even if the noise reduction means is omitted, when the holding time Ty1, the elapsed time Te, the setting values Is1 and Is2 of the current, and the like described above are set within the preferable ranges thereof, it is possible to improve the taste modification effect while suppressing the current value to be relatively small as compared with a case where the holding time Ty1, the elapsed time Te, the setting values Is1 and Is2 of the current, and the like are set out of the preferable ranges.

The food or drink that is a target of taste presentation according to the present invention may include various foods and drinks as long as the current for generating the electrical stimulation can pass therethrough. Although any food or drink containing moisture can cause the current to flow therethrough, it is preferable to use, as the target, a food or drink containing moisture in an amount of 20% by weight or more, more preferably 25% by weight or more, and still more preferably 30% by weight or more. The effect of modifying the taste can be exhibited in various foods and drinks as long as a taste component is contained. For example, when a salty taste of a food or drink containing salt is to be enhanced, the present invention can be preferably applied to a food or drink containing salt in an amount of 0.05% by weight or more, and the present invention can be applied to a food or drink containing salt in an amount of more preferably 0.1% by weight or more, and still more preferably 0.15% or more. As specific foods or drinks, noodles such as ramen, soups, meat dishes and processed meat products such as sausages and fried chicken, fish dishes and processed fish products such as grilled fish and fish roe, egg dishes and processed egg products, bean curd dishes and processed bean curd products, cheese, cooked processed products, vegetable dishes and processed vegetable products, powdered products, fried products, foods served in hot pot, stewed dishes such as curry, alcoholic beverages such as beer and wine, near water beverages, sports beverages (isotonic beverages), fruit juice beverages, milk beverages, vegetable juice beverages, acidic beverages, carbonated beverages, whey beverages, coffee beverages, black tea beverages, oolong tea beverages, barley tea beverages, energy drinks, and non-alcoholic beverages such as beer-taste beverages can be used as the target of taste modification according to the present invention. The taste to be modified is not limited to the salty taste, and various tastes such as a sweet taste, a sour taste, a savory taste, a bitter taste, an astringent taste, a pungent taste, a fat taste, a sparkling taste, and an alcohol taste can be modified and presented according to the present invention.

An effect of enhancing the taste (for example, salty taste), degrees of strength of the sense of discomfort and the electric taste in the present invention can be easily and clearly decided by trained panelists. General methods can be used as evaluation criteria and the way of summarizing evaluations among the panelists. In the present invention, the number of panelists who evaluate the taste (for example, salty taste) enhancing effect and the degrees of strength of the sense of discomfort and the electric taste may be one. However, from the viewpoint of obtaining more objective evaluations, a lower limit of the number of panelists can be, for example, two or more and preferably three or more. From the viewpoint of more easily performing an evaluation test, an upper limit of the number of panelists may be, for example, twenty or less, or ten or less. Evaluations of the taste (for example, salty taste) enhancing effect and the degrees of strength of the sense of discomfort and the electric taste when there are two or more panelists may adopt averages of evaluations of all the panelists regarding the taste (for example, salty taste) enhancing effect and the degrees of strength of the sense of discomfort and the electric taste for a food or drink sample. When integer evaluation points are given to the respective evaluation criteria, an average value of evaluation points of all the panelists may be adopted as the evaluation of each of the taste (for example, salty taste) enhancing effect and the degrees of strength of the sense of discomfort and the electric taste. When the average value of the evaluation points is adopted as described above, a value obtained by rounding the average value to one decimal place or two decimal places (preferably two decimal places) may be adopted. Incidentally, when there are two or more panelists, in order to reduce variations in the evaluations of the respective panelists, it is preferable to perform work of standardizing the evaluation criteria such that the evaluation criteria of the respective panelists are as uniform as possible before performing an actual sensory evaluation test. As an example of such standardization work, after the respective panelists evaluate the taste strength, and the degrees of strength of the sense of discomfort and the electric taste for a plurality of types of food or drink samples whose taste (for example, salty taste) enhancing effects are known, evaluation points thereof are compared, and it is confirmed that there is no large discrepancy in the evaluation criteria of the respective panelists. Further, it is preferable to set a standard deviation of the evaluation of each of the taste (for example, salty taste) enhancing effect and the degrees of strength of the sense of discomfort and the electric taste by the respective panelists to be within 0.5 when the evaluation point in any one of a plurality of stages (any of five stages of, for example, 1, 2, 3, 4, and 5) is given by such standardization work regarding the evaluation criteria in advance.

### EXAMPLES

Next, tests performed to confirm the effects of the present invention will be described. Test conditions are as follows.

### (1) Regarding Test Conditions

### [Test Device]

For the tests, a device including the electrodes configured as illustrated in FIG. 1 and the electrical stimulation generation unit configured as illustrated in FIG. 3 was used as a test device. However, an AC commercial power source of AC 100 V was used as the external power source, and AC was converted into DC of 20 V and 2.25 A by an AC adapter and supplied to the booster circuit.

In the test device, stainless metal was used for both the first electrode and the second electrode. The first electrode was configured to be held by a hand of a subject, and the second electrode was connected to the conductive muddler. The conductive muddler was disposed in a cup containing a food or drink sample. As for the noise reduction units, an inductor with a part number LQM21PN4R7MGRD manufactured by Murata Manufacturing Co., Ltd., was used as the noise reduction unit 17A intended to reduce a noise component derived from the external power source, and an inductor of MLZ2012 type with a part number MLZ2012N101LT000 manufactured by TDK Corporation was used as the noise reduction unit 17B corresponding to the internal oscillation frequency of the booster circuit.

### [Food or Drink Sample]

The food or drink sample to be ingested was prepared as follows.
Tasting sample: A commercially available low-salt instant miso soup was prepared with 1.5 times a specified amount of hot water. The salt equivalent per meal was 1.2 g, and the salt equivalent of the tasting sample was 0.5% by weight.
Tasting condition: The tasting sample at normal temperature (20 to 25° C.) was tasted.

### [Evaluation Method]

Subject

Three subjects confirmed to be capable of discriminating a difference in strength of a salty taste at a salt concentration of 0.03% by weight by a preliminary test were selected. Incidentally, before performing the actual sensory evaluation test, the appropriateness of the selected subjects were confirmed as follows. That is, in order to reduce variations in evaluations of the respective subjects, the salty taste enhancing effect as taste strength and the degrees of strength of the sense of discomfort and the electric taste were evaluated using a plurality of types of known food or drink samples. Taste presentation was performed with an electrode for electrical stimulation in contact with the tongue. Evaluation points of the subjects were compared to confirm that there is no large discrepancy in evaluation criteria among the subjects. Furthermore, it was also confirmed that a standard deviation of evaluations of each of the salty taste enhancing effect and the degrees of strength of the sense of discomfort and the electric taste by the respective subjects was within 0.5.
Ingestion Method and Evaluation Method of Food or Drink

The respective subjects were made to ingest the tasting sample using the test device. The ingestion was performed by a technique in which the tasting sample was placed in a cup in which the conductive muddler was disposed and each of the subjects directly puts the mouth onto the cup. During the ingestion, a current for taste stimulation was supplied between the electrodes from the electrical stimulation generation unit, and the respective subjects were made to give scores according to criteria in Tables 1 to 3 below for each of the salty taste enhancing effect, an electrified feeling, and the electric taste, thereby acquiring sensory evaluations. The salty taste enhancing effect was evaluated as an index indicating a degree of the taste modification effect. The electrified feeling is an unnatural sensation that may be felt in a body part close to the electrodes when the current flows, and is evaluated as an index indicating the degree of the sense of discomfort. After the sensory evaluations were obtained, a value obtained by rounding an average value of evaluation points of all the subjects to three decimal places was obtained as an average value of evaluation results. Further, based on free opinions from the subjects, a problem due to a time difference from when the food or drink was put in the mouth to when a modified taste was sensed, that is, the above-described unnaturalness of timing was also evaluated as another index indicating the degree of the sense of discomfort.

TABLE 1

| Score | Criterion |
|---|---|
| 5 | Increase by 40% or more |
| 4 | Increase by 30 to 40% |
| 3 | Increase by 20 to 30% |
| 2 | Increase by 10 to 20% |
| 1 | Increase by 1 to 10% |
| 0 | Unchanged |

TABLE 2

| Score | Criterion |
|---|---|
| 5 | Intolerable |
| 4 | It is tolerable in case of test, |
| | but it is intolerable in daily life |
| 3 | It is bothersome but is not so |
| | intolerable in daily life |
| 2 | It is not so bothersome. |
| 1 | No feeling |

TABLE 3

| Score | Criterion |
|---|---|
| 5 | Electric taste is sensed when test food is put in |
| | mouth and after test food is swallowed. |
| 4 | Electric taste is slightly sensed when test food is |
| | put in mouth and after test food is swallowed. |
| 3 | If pointed out, electric taste is sensed when test |
| | food is put in mouth and after test food is |
| | swallowed. Unless pointed out, it is not bothersome. |
| 2 | If pointed out, electric taste may be felt when test |
| | food is put in mouth and after test food is |
| | swallowed. Unless pointed out, it is not noticed. |
| 1 | No electric taste is sensed. |

Regarding the salty taste enhancing effect in Table 1, three or more points were evaluated as “effective”. Regarding the electrified feeling in Table 2 and the electric taste in Table 3, three or less points were evaluated as “no problem”.

(2) Confirmation of Influence of Holding Time of Cathode Stimulation and Elapsed Time until Start of Anode Stimulation

A test was performed to confirm the influence of the holding time of the cathode stimulation and the elapsed time until the start of the anode stimulation on the salty taste enhancing effect and the like. In the test, as illustrated in FIG. 4, the current control was applied to perform the cathode stimulation first and then reverse the polarity to the anode stimulation, and the increase time Tx1, the holding time Ty1, and the reversal time Tx2 were set as in Test Examples 1 to 5 in Table 4, and the salty taste enhancing effect and the electrified feeling in each of Test Examples were evaluated according to the above evaluation method. In all of Test Examples 1 to 5, absolute values of the setting values Is1 and Is2 of the current were set to 0.50 mA, and the holding time Ty2 in the anode stimulation was set to 0.80 seconds.

TABLE 4

| | Current setting | | | | Salty taste | | | | Electrified | | | | |
|---|---|---|---|---|---|---|---|---|---|---|---|---|---|
| | condition | | | | enhancing effect | | | | feeling | | | | |
| | Increase | Holding | Reversal | Elapsed | Average | | | | Average | | | | Difference |
| | time Tx1 | time Ty1 | time Tx2 | time Te | value a | P1 | P2 | P3 | value b | P1 | P2 | P3 | a − b |
| Test | 0.30 | 0.50 | 0.40 | 1.20 | 4.00 | 4 | 4 | 4 | 1.67 | 2 | 2 | 1 | 2.33 |
| Example | seconds | seconds | seconds | seconds | | | | | | | | | |
| 1 | | | | | | | | | | | | | |
| Test | 0.15 | 0.25 | 0.20 | 0.60 | 2.67 | 3 | 3 | 2 | 2.67 | 2 | 3 | 3 | 0.00 |
| Example | seconds | seconds | seconds | seconds | | | | | | | | | |
| 2 | | | | | | | | | | | | | |
| Test | 0.15 | 0.50 | 0.20 | 0.85 | 4.00 | 4 | 4 | 4 | 3.00 | 3 | 3 | 3 | 1.00 |
| Example | seconds | seconds | seconds | seconds | | | | | | | | | |
| 3 | | | | | | | | | | | | | |
| Test | 0.15 | 0.50 | 0.30 | 0.95 | 4.00 | 4 | 4 | 4 | 2.00 | 2 | 2 | 2 | 2.00 |
| Example | seconds | seconds | seconds | seconds | | | | | | | | | |
| 4 | | | | | | | | | | | | | |
| Test | 0.20 | 0.50 | 0.20 | 0.90 | 4.00 | 4 | 4 | 4 | 1.33 | 1 | 2 | 1 | 2.67 |
| Example | seconds | seconds | seconds | seconds | | | | | | | | | |
| 5 | | | | | | | | | | | | | |

Evaluation results of the salty taste enhancing effect and the electrified feeling in Test Examples 1 to 5 are as shown in Table 4. The evaluation results indicate the average values of evaluation scores of the subjects in two decimal places in addition to the individual evaluation scores of the subjects. Three subjects were denoted P1 to P3. The higher the score, the salty taste enhancing effect is highly evaluated as the score is higher, and the electrified feeling is highly evaluated as the score is lower. Therefore, a difference between the average values of the both was obtained and shown in the rightmost column of Table 4. The difference between the average values can be used as an index indicating that both exhibition of the salty taste enhancing effect and suppression of the electrified feeling are more compatible as the value thereof is larger.

When Test Examples 1 to 5 are compared, it can be confirmed that, the average value of the salty taste enhancing effect was 4 in Test Examples 1 and 3 to 5 in which the holding time Ty1 in the cathode stimulation was 0.50 seconds, and a sufficient salty taste enhancing effect was obtained by performing the cathode stimulation first although the setting value Is2 of the current in the anode stimulation was a relatively small value of 0.50 mA. On the other hand, in Test Example 2 in which the holding time Ty1 was set to 0.25 seconds, the average value of the salty taste enhancing effect was less than 3, and a sufficient effect of performing the cathode stimulation first was not necessarily obtained. Regarding the electrified feeling, in all of Test Examples 1 to 5, the average values of the evaluation scores is 3 points or less set as the criterion for “no problem”. However, when a degree of the exhibition of the salty taste enhancing effect and an effect of suppressing the electrified feeling are collated using the difference between the average values as an index, Test Example 2 has the minimum difference between the average values. It is understood that the reason is that the holding time Ty1 is short. From the above, when the holding time Ty1 in the cathode stimulation is 0.30 seconds or more, which is longer than that in Test Example 2, it can be determined that the salty taste enhancing effect can be exhibited while the setting value Is2 of the current in the anode stimulation is suppressed to be relatively small, and further, that the electrified feeling can be moderately suppressed. When the holding time Ty1 is set to 0.50 seconds or more as in Test Examples 1 and 3 to 5, the effect of performing the cathode stimulation first can be more reliably exhibited. From the above, it is determined that the holding time Ty1 is preferably set to 0.30 seconds or more in consideration of 0.25 seconds in Test Example 2 with some margin. It is presumed that when the holding time Ty1 is set to 0.35 seconds or more, more preferably 0.40 seconds or more, and most preferably 0.50 seconds or more, the electrified feeling, that is, the unnatural sensation that may be felt near the electrodes can be suppressed to an extent that there is no problem, and both the salty taste enhancing effect and the electrified feeling can be achieved more reliably.

The influence of the elapsed time Te was evaluated as the unnaturalness of the timing based on the free opinions from the subjects. As a result, regarding all of Test Examples 1 to 5 including Test Example 1 having the maximum elapsed time Te, it was confirmed that there is no opinion from the subjects and the influence was within an acceptable range without any particular problem. As long as the holding time Ty1 is secured to be 0.30 seconds or more as described above, it is determined that the elapsed time Te is preferably 1.30 seconds or less in consideration of 1.20 seconds of Test Example 1 with some margin. Therefore, it is presumed that there is no time difference (unnaturalness of timing) between the timing at which the food or drink is put in the mouth and the taste sensing and both the salty taste enhancing effect and the electrified feeling can be more reliably achieved when the elapsed time Te is set to 1.20 seconds or less, and more preferably 1.00 seconds or less.

Further, when the elapsed time Te is set to the above upper limit or less, the upper limit of the holding time Ty1 is within a range not exceeding the elapsed time Te, and when considering that the increase time Tx1 was 0.15 seconds and the reversal time Tx2 was 0.20 seconds in Test Example 4, it is presumed that there is no time difference (unnaturalness of timing) between the timing at which the food or drink is put in the mouth and the taste sensing and both the salty taste enhancing effect and the electrified feeling can be achieved, if the upper limit of the holding time Ty1 is set to be equal to or less than 0.95 seconds, which is a value obtained by subtracting the increase time Tx1 and the reversal time Tx2 from the upper limit of 1.30 seconds of the holding time Te, preferably to 0.85 seconds or less, more preferably to 0.75 seconds or less, and most preferably to 0.65 seconds or less. It is presumed that the lower limit of the elapsed time Te may be sufficiently set to 0.70 seconds or more in consideration of 0.60 seconds in Test Example 2 with some margin, and may be more reliable by preferably setting to 0.85 seconds or more in Test Example 3 as a guide.

The increase time Tx1 does not need to be strictly limited considering that there is a difference in the difference between the average values even when all of Test Examples 2 to 4 were set to 0.15 seconds, and the difference between the average values was good in Test Example 1 having the maximum value, but it is presumed that the lower limit thereof may be sufficiently set to about 0.10 seconds or more, preferably 0.15 seconds or more, and more preferably 0.20 seconds or more, and the upper limit thereof may be sufficiently set to 0.50 seconds or less. The reversal time Tx2 does not need to be strictly limited considering that there is a difference in the difference between the average values even when all of Test Examples 2, 3, and 5 were set to 0.20 seconds, and the difference between the average values was good in Test Example 1 having the maximum value, but it is presumed that it is sufficient to set the reversal time Tx2 within a range of 0.20 seconds or more and 0.50 seconds or less.

Incidentally, an attempt was made to evaluate the electric taste in Test Examples 1 to 5, but an average value of evaluation scores exceeded 3 in all of Test Examples, and there was no difference between Test Examples. Therefore, there is room for further study on various parameters set in Test Examples 1 to 5 in terms of reduction of the electric taste. On the other hand, in consideration of the comparison among Test Examples 1 to 5, it can be determined that the purpose of exhibiting the salty taste enhancing effect to such an extent as to be sensed by the user while suppressing the setting value of the current in the anode stimulation to such an extent that the electrified feeling of the user is alleviated or mitigated can be achieved by setting at least the holding time Ty1 to 0.30 seconds or more as described above. Furthermore, if the elapsed time Te is set to 1.30 seconds or less, the effect of suppressing the unnaturalness of timing can be expected.

### (3) Confirmation of Influence of Setting Values of Current

A test was performed to confirm the influence of the setting values Is1 and Is2 of the current in the cathode stimulation and the anode stimulation. In the test, as illustrated in FIG. 4, the current control was applied to perform the cathode stimulation first and then reverse the polarity to the anode stimulation, and the setting values Is1 and Is2 of the current were set as shown in Test Examples 6 to 13 in Table 5. Incidentally, the setting values Is1 and Is2 in Table 5 are absolute values. Three subjects are denoted P1 to P3. In Test Examples 6 to 13, the increase time Tx1 of the current of the cathode stimulation was set to 0.30 seconds, the holding time Ty1 of the current was set to 0.50 seconds, the reversal time Tx2 was set to 0.40 seconds, and the holding time Ty2 of the current of the anode stimulation was set to 0.80 seconds. These setting values are the same as those in Test Example 1 in Table 4. In Test Example 6, since the absolute values of the setting values Is1 and Is2 of the current are both 0.50 mA, conditions match those of Test Example 1.

TABLE 5

| | Current | | | | | | | | | | | | | | |
|---|---|---|---|---|---|---|---|---|---|---|---|---|---|---|---|
| | setting value | | Salty taste | | | | Electrified | | | | | | | | |
| | Setting | Setting | enhancing effect | | | | feeling | | | | Electric taste | | | | |
| | value | value | Average | | | | Average | | | | Average | | | | Difference |
| | Is1 | Is2 | value a | P1 | P2 | P3 | value b | P1 | P2 | P3 | value c | P1 | P2 | P3 | a − b − c |
| Test | 0.50 mA | 0.50 mA | 4.00 | 4 | 4 | 4 | 1.67 | 2 | 2 | 1 | 3.67 | 3 | 5 | 3 | −1.33 |
| Example | | | | | | | | | | | | | | | |
| 6 | | | | | | | | | | | | | | | |
| Test | 0.50 mA | 0.40 mA | 4.00 | 4 | 4 | 4 | 1.67 | 2 | 2 | 1 | 2.67 | 2 | 3 | 3 | −0.33 |
| Example | | | | | | | | | | | | | | | |
| 7 | | | | | | | | | | | | | | | |
| Test | 0.50 mA | 0.20 mA | 3.67 | 4 | 3 | 4 | 1.67 | 2 | 2 | 1 | 2.33 | 2 | 3 | 2 | −0.33 |
| Example | | | | | | | | | | | | | | | |
| 8 | | | | | | | | | | | | | | | |
| Test | 0.50 mA | 0.10 mA | 3.00 | 3 | 3 | 3 | 1.67 | 2 | 2 | 1 | 1.33 | 1 | 2 | 1 | 0.00 |
| Example | | | | | | | | | | | | | | | |
| 9 | | | | | | | | | | | | | | | |
| Test | 0.30 mA | 0.30 mA | 3.33 | 4 | 3 | 3 | 2.33 | 3 | 2 | 2 | 3.00 | 3 | 3 | 3 | −2.00 |
| Example | | | | | | | | | | | | | | | |
| 10 | | | | | | | | | | | | | | | |
| Test | 0.30 mA | 0.20 mA | 3.00 | 3 | 3 | 3 | 1.67 | 2 | 2 | 1 | 2.00 | 2 | 2 | 2 | −0.67 |
| Example | | | | | | | | | | | | | | | |
| 11 | | | | | | | | | | | | | | | |
| Test | 0.10 mA | 0.10 mA | 1.67 | 2 | 1 | 2 | 1.00 | 1 | 1 | 1 | 1.67 | 1 | 2 | 2 | −1.00 |
| Example | | | | | | | | | | | | | | | |
| 12 | | | | | | | | | | | | | | | |
| Test | 0.10 mA | 0.05 mA | 1.67 | 2 | 1 | 2 | 1.00 | 1 | 1 | 1 | 1.00 | 1 | 1 | 1 | −0.33 |
| Example | | | | | | | | | | | | | | | |
| 13 | | | | | | | | | | | | | | | |

Evaluation results of the salty taste enhancing effect, the electrified feeling, and the electric taste in Test Examples 6 to 13 are as shown in Table 5. The evaluation results indicate the average values of evaluation scores of each subject in two decimal places in addition to the individual evaluation scores of each subject. The higher the score, the salty taste enhancing effect is highly evaluated as the score is higher, and the electrified feeling and the electric taste are highly evaluated as the score is lower. Therefore, as a difference among the average values of these evaluation results, a value was obtained by subtracting the average values of the electrified feeling and the electric taste from the average value of the salty taste enhancing effect and shown in the rightmost column of Table 5. The difference among the average values in Table 5 can be used as an index indicating that both exhibition of the salty taste enhancing effect and suppression of the electrified feeling and the electric taste are more compatible as the value thereof is larger.

As is apparent from Table 5, in Test Example 6 in which the absolute values of the setting values Is1 and Is2 of the current in the cathode stimulation and the anode stimulation were set to be equal to each other, the absolute values being 0.5 mA, an evaluation that the salty taste enhancing effect is “effective” and there is “no problem” for the electrified feeling is obtained, but there is no sufficient reducing effect in relation to the electric taste. On the other hand, in Test Examples 10 and 11 in which the absolute values of the setting values Is1 and Is2 were set to be equal to each other, since the absolute values were set to be as small as 0.30 mA and 0.10 mA, respectively, not only the electrified feeling but also the electric taste is improved to be equal to or less than 3, which is the criterion for “no problem”. Further, in Test Examples 7, 8, 9, 11, and 13 in which the absolute value of the setting value Is1 in the cathode stimulation was set to be larger than the absolute value of the setting value Is2 in the anode stimulation, the salty taste enhancing effect slightly decreases with a decrease in the setting value Is2, the evaluation scores for the electrified feeling and the electric taste were sufficiently suppressed to be smaller than 3 as the criterion for “no problem”. In view of these results, when the absolute values of the setting values Is1 and Is2 of the current are set to be equal to each other and to 0.30 mA or less, or when the absolute value of the setting value Is1 in the cathode stimulation is set to be larger than the absolute value of the setting value Is2 in the anode stimulation, it can be determined that it is possible to suppress the electrified feeling and the electric taste to such an extent that there is no problem while exhibiting the salty taste enhancing effect to an appropriate extent.

When the magnitude relationship of the setting values is set as Is1=Is2, there is “no problem” for the electrified feeling and the electric taste if the absolute value of the setting value Is1 of the current is set within the range of being equal to or less than 0.30 mA, which is the setting value Is1 in Test Example 10, and the electric taste exceeds the range of “no problem” if the absolute value is set to 0.50 mA. Therefore it is presumed that the degree of the electric taste has “no problem” if the absolute value is set to 0.30 mA or less. Therefore, when the setting values are Is1=Is2, the absolute values of the setting values Is1 and Is2 are preferably set to 0.30 mA or less so that the salty taste enhancing effect, the electrified feeling, and the electric taste can be satisfied. It is possible to expect suitable effects when the lower limit of the absolute values of the setting values Is1 and Is2 is set to 0.25 mA or more in consideration of environmental conditions such as the fact that the “salty taste enhancing effect” fell below 3, which is the level of “effective” in Test Example 12 in which 0.10 mA is set, individual differences of users, a degree of the intended taste modification effect, and a difference in target food or drink.

Further, when the magnitude relationship of the setting values Is1 and Is2 is set to Is1>Is2, it is understood that the absolute value of the setting value Is1 of the current in the cathode stimulation is reliable in the range of being equal to or less than 0.50 mA, which is the setting value Is1 in Test Examples 6 to 9, from the viewpoint of the exhibition of the effects. However, considering environmental conditions such as individual differences of users, the degree of the intended taste modification effect, and a difference in target food or drink, there is a possibility that suitable effects can be obtained even if the absolute value of the setting value Is1 is 0.70 mA or less, and further 1.00 mA or less. It is possible to expect suitable effects even if the lower limit of the absolute values of the setting values Is1 and Is2 is set to 0.05 mA or more, and further to 0.01 mA or more as an example.

Various aspects of the present invention derived from each of the above-described embodiment, modified examples, and examples will be described below. Incidentally, in the following description, to facilitate understanding of each aspect of the present invention, corresponding constituent elements illustrated in the accompanying drawings are added in parentheses, which however does not limit the present invention to the illustrated mode.

A taste presentation device (10) according to an aspect of the present invention includes: a first electrode (11) and a second electrode (12) provided to be capable of forming an electric circuit between a food or drink (2) to be ingested by a user and a body of the user; and electrical stimulation generation means (13) supplying a current to generate electrical stimulation between the first electrode and the second electrode, wherein the electrical stimulation generation means is provided to start the supply of the current in a state of cathode stimulation in which the second electrode is a cathode, and then to change a direction of the current so as to switch to a state of anode stimulation in which the second electrode is an anode, and in the cathode stimulation, a holding time (Ty1) during which the current is to be held at a setting value (Is1) of the current in the cathode stimulation is set to 0.30 seconds or more.

A taste presentation method according to an aspect of the present invention includes: a process of providing a first electrode (11) and a second electrode (12) to be capable of forming an electric circuit between a food or drink (2) to be ingested by a user and a body of the user; and a process of supplying a current to generate electrical stimulation between the first electrode and the second electrode, wherein, in the process of supplying the current, the supply of the current is started in a state of cathode stimulation in which the second electrode is a cathode, and then a direction of the current is changed so as to switch to a state of anode stimulation in which the second electrode is an anode, and in the cathode stimulation, a holding time (Ty1) during which the current is to be held at a setting value (Is1) of the current in the cathode stimulation is set to 0.30 seconds or more.

There is a tendency that the cathode stimulation suppresses a sense of discomfort and an electric taste sensed by the user to be relatively weak but has a weak taste modification effect, and the anode stimulation has a relatively strong taste modification effect but induces the sense of discomfort and the electric taste. The sense of discomfort and the electric taste during the anode stimulation increase as a current value during the anode stimulation increases. Further, the taste modification effect tends to be more effectively exhibited as the amount of change in the current at the start of the anode stimulation is larger. Therefore, if the anode stimulation is performed by reversing the polarity after the cathode stimulation is performed first, it is possible to secure a large amount of change in the current value during the reversal to enhance the taste modification effect, and it is possible to suppress the increase in the sense of discomfort and the electric taste by suppressing a setting value of the current during the anode stimulation to be relatively small. Furthermore, an effect of performing the cathode stimulation first is affected by the holding time for which the current value is to be held at a predetermined setting value in the cathode stimulation, and the effect of performing the cathode stimulation first is lowered as the holding time is shorter. Therefore, by setting the holding time to 0.30 seconds or more, the effect of performing the cathode stimulation first can be exhibited, and the taste modification effect can be exhibited so that the user can feel the taste modification effect while suppressing the current value in the anode stimulation to be small.

In the taste presentation device of the above aspect, the holding time in the cathode stimulation may be set to 0.50 seconds or more. Accordingly, the effect of performing the cathode stimulation first can be more reliably exhibited.

An elapsed time (Te) from the start of the supply of the current in the cathode stimulation to when the current reaches a setting value (Is2) of the current in the anode stimulation by the anode stimulation may be set to 1.30 seconds or less. The taste modification effect is exhibited after the anode stimulation is started, but when the elapsed time until the current reaches the setting value in the anode stimulation is prolonged, the exhibition of the taste modification effect is delayed with respect to the user's eating and drinking behavior, which causes the user to feel the sense of discomfort. Therefore, by setting the elapsed time to 1.30 seconds or less, it is possible to exhibit the taste modification effect at a time in accordance with the user's eating and drinking behavior, and to reduce or mitigate the sense of discomfort.

In the taste presentation device of the above aspect, an absolute value of the setting value (Is1) of the current in the cathode stimulation may be set to be larger than an absolute value of a setting value (Is2) of the current in the anode stimulation. When such a magnitude relationship is set, the taste modification effect can be more reliably exhibited by increasing the amount of change in the current value at the time of the polarity reversal while suppressing the setting value of the current during the anode stimulation to be relatively small.

When the absolute value of the setting value of the current in the cathode stimulation is larger than the absolute value of the setting value of the current in the anode stimulation, the absolute value of the setting value (Is1) of the current in the cathode stimulation may be set to 0.50 mA or less. Accordingly, the absolute value of the current at the time of anode stimulation is limited to be smaller than 0.50 mA, so that it is possible to enhance an effect of suppressing the sense of discomfort and electric taste during the anode stimulation.

Alternatively, absolute values of setting values (Is1, Is2) of the current in the cathode stimulation and the anode stimulation may be set to be equal to each other and may be set to 0.25 mA or more. Further, absolute values of setting values (Is1, Is2) of the current in the cathode stimulation and the anode stimulation may be set to be equal to each other and may be set to 0.30 mA or less. According to these embodiments, it is possible to reliably exhibit the taste modification effect while suppressing the sense of discomfort and the electric taste.

When the elapsed time is set to 1.30 seconds or less, absolute value of the setting value of the current in each of the cathode stimulation and the anode stimulation may be set to 0.50 mA or less, and an increase time (Tx1) from the start of the supply of the current in the cathode stimulation to when the current reaches the setting value may be set to 0.10 seconds or more, and a reversal time (Tx2) during which the current is changed from the setting value in the cathode stimulation to the setting value in the anode stimulation may be set to 0.20 seconds or more. Accordingly, it is possible to suppress a rapid change in the current during the cathode stimulation and during the reversal from the cathode stimulation to the anode stimulation, and to suppress or avoid the generation of the sense of discomfort caused by the change in the current.

In the taste presentation device of the above aspect, the first electrode may be provided to be in contact with the body of the user, and the second electrode may be provided to be in contact with the food or drink. By disposing the electrodes in this manner, the electric circuit can be formed between the food or drink and the body of the user at the time of ingestion of the food or drink.

The electrical stimulation generation means may include noise reduction means (17A, 17B) reducing a noise component in the current. By the noise reduction means reducing the noise component in the current supplied between the electrodes, it is possible to suppress the influence of the noise component on the sense of discomfort and the electric taste and to enhance the intended taste modification effect.

The noise reduction means (17A) may be provided to reduce a noise component derived from a power source. When the electrical stimulation generation means include a booster circuit (14), the noise reduction means (17B) may be provided to reduce a noise component corresponding to an internal oscillation frequency in the booster circuit. According to these embodiments, it is possible to effectively reduce the noise components that cause deterioration in the sense of discomfort and the electric taste.

1. A taste presentation device, comprising:
a first electrode and a second electrode configured to be capable of forming an electric circuit between a food or drink to be ingested by a user and a body of the user; and
an electrical stimulation generator, configured to supply a current to generate electrical stimulation between the first electrode and the second electrode,
wherein the electrical stimulation generator is configured to start the supply of the current in a state of cathode stimulation in which the second electrode is a cathode, and then to change a direction of the current so as to switch to a state of anode stimulation in which the second electrode is an anode, and
wherein, in the cathode stimulation, a holding time, during which the current is to be held at a setting value of the current in the cathode stimulation, is set to 0.30 seconds or more.

2. The taste presentation device of claim 1, wherein the holding time in the cathode stimulation is set to 0.50 seconds or more.

3. The taste presentation device of claim 1, wherein an elapsed time from the start of the supply of the current in the cathode stimulation to when the current reaches a setting value of the current in the anode stimulation by the anode stimulation is set to 1.30 seconds or less.

4. The taste presentation device of claim 1, wherein an absolute value of the setting value of the current in the cathode stimulation is set to be larger than an absolute value of a setting value of the current in the anode stimulation.

5. The taste presentation device of claim 4, wherein the absolute value of the setting value of the current in the cathode stimulation is set to 0.50 mA or less.

6. The taste presentation device of claim 1, wherein absolute values of setting values of the current in the cathode stimulation and the anode stimulation are set to be equal to each other and are set to 0.25 mA or more.

7. The taste presentation device of claim 1, wherein absolute values of setting values of the current in the cathode stimulation and the anode stimulation are set to be equal to each other and are set to 0.30 mA or less.

8. The taste presentation device of claim 3, wherein absolute value of the setting value of the current in each of the cathode stimulation and the anode stimulation is set to 0.50 mA or less, and
wherein an increase time from the start of the supply of the current in the cathode stimulation to when the current reaches the setting value is set to 0.10 seconds or more, and a reversal time during which the current is changed from the setting value in the cathode stimulation to the setting value in the anode stimulation is set to 0.20 seconds or more.

9. The taste presentation device of claim 1, wherein the first electrode is configured to be in contact with the body of the user, and
wherein the second electrode is configured to be in contact with the food or drink.

10. The taste presentation device of claim 1, wherein the electrical stimulation generator comprises a noise reducer configured to reduce a noise component in the current.

11. The taste presentation device of claim 10, wherein the noise reducer is configured to reduce a noise component derived from a power source.

12. The taste presentation device of claim 10, wherein the electrical stimulation generator comprises a booster circuit, and
wherein the noise reducer is configured to reduce a noise component corresponding to an internal oscillation frequency in the booster circuit.

13. A taste presentation method, comprising:
providing a first electrode and a second electrode to be capable of forming an electric circuit between a food or drink to be ingested by a user and a body of the user; and
supplying a current to generate electrical stimulation between the first electrode and the second electrode,
wherein, in the supplying of the current, a supply of the current is started in a state of cathode stimulation in which the second electrode is a cathode, and then a direction of the current is changed so as to switch to a state of anode stimulation in which the second electrode is an anode, and
wherein, in the cathode stimulation, a holding time during which the current is to be held at a setting value of the current in the cathode stimulation is set to 0.30 seconds or more.

14. The taste presentation device of claim 2, wherein the first electrode is configured to be in contact with the body of the user, and
wherein the second electrode is configured to be in contact with the food or drink.

15. The taste presentation device of claim 3, wherein the first electrode is configured to be in contact with the body of the user, and
wherein the second electrode is configured to be in contact with the food or drink.
